(19) United States
(12) Patent Application Publication
(10) Pub. No.: US 2019/0205336 A1
(43) Pub. Date: Jul. 4, 2019

(54) INFORMATION PROCESSING APPARATUS, INFORMATION PROCESSING METHOD, INFORMATION PROCESSING SYSTEM, AND NON-TRANSITORY COMPUTER-READABLE MEDIUM

(71) Applicant: CANON KABUSHIKI KAISHA, Tokyo (JP)

(72) Inventors: Ryosuke Mizuno, Matsudo-shi (JP); Nobu Miyazawa, Yokohama-shi (JP); Yukari Nakashoji, Tokyo (JP)

(21) Appl. No.: 16/299,837

(22) Filed: Mar. 12, 2019

Related U.S. Application Data
(63) Continuation of application No. PCT/JP2017/040431, filed on Nov. 9, 2017.

(30) Foreign Application Priority Data
Nov. 22, 2016 (JP) 2016-227101

(51) Int. Cl. G06F 16/583; A61B 5/00; G06F 16/58

(52) U.S. Cl. CPC G06F 16/583; G06F 16/5866; A61B 5/0095

(57) ABSTRACT

An information processing apparatus determines, on a basis of information related to types of photoacoustic images of a plurality types which are generated on a basis of a photoacoustic signal, whether or not the photoacoustic images are output to an external apparatus, and outputs the photoacoustic images and meta data related to the photoacoustic images to the external apparatus in a case where it is determined that the photoacoustic images are output to the external apparatus and outputs only the meta data among the photoacoustic images and meta data to the external apparatus in a case where it is determined that the photoacoustic images are not output to the external apparatus.

### CROSS-REFERENCE TO RELATED APPLICATIONS

This application is a Continuation of International Patent Application No. PCT/JP2017/040431, filed Nov. 9, 2017, which claims the benefit of Japanese Patent Application No. 2016-227101, filed Nov. 22, 2016, both of which are hereby incorporated by reference herein in their entirety.

### TECHNICAL FIELD

The present invention relates to an information processing apparatus, an information processing method, an information processing system, and a program.

### BACKGROUND ART

As a technique for imaging an internal state of a subject in a low invasive manner, research on photoacoustic imaging has been advanced. The photoacoustic imaging is a technique for obtaining an optical characteristic value distribution in a living matter in a high resolution by using a characteristic of an ultrasonic wave involving less scattering in the living matter than light. NPL 1 discloses that image reconstruction is performed on the basis of a signal of an ultrasonic wave generated inside a subject, and a distribution of an acoustic pressure inside the subject can be obtained. Furthermore, it has been proposed that an absorption coefficient of a substance inside the subject is imaged on the basis of the distribution of the acoustic pressure, and images of various types representing a substance component ratio inside the subject and information related to a function such as metabolism are obtained.

### CITATION LIST

### Non Patent Literature

NPL 1 M, Xu, L. V. Wang “Photoacoustic imaging in biomedicine”, Review of scientific instruments, 77, 041101 (2006)

Various mages can be obtained from a certain signal of the ultrasonic wave generated inside the subject by using an imaging apparatus using the photoacoustic imaging. However, in a case where all of the various images obtained by a single examination are saved or output to an external apparatus, there is a fear that a capacity for saving or performing a communication is squeezed.

### SUMMARY OF INVENTION

An information processing apparatus according to an embodiment of the present invention includes a determination unit configured to determine, on a basis of information related to types of photoacoustic images of a plurality types which are generated on a basis of a photoacoustic signal obtained by irradiating a subject with light, whether or not the photoacoustic images are output to an external apparatus, and an output unit configured to output the photoacoustic images and meta data related to the photoacoustic images to the external apparatus in a case where the determination unit determines that the photoacoustic images are output to the external apparatus, and output only the meta data among the photoacoustic images and meta data to the external apparatus in a case where the determination unit determines that the photoacoustic images are not output to the external apparatus.

Further features of the present invention will become apparent from the following description of exemplary embodiments with reference to the attached drawings.

### BRIEF DESCRIPTION OF DRAWINGS

FIG. 1 illustrates an example of a functional configuration of an information processing apparatus according to a first embodiment.

FIG. 2A illustrates an example of information obtained by the information processing apparatus according to the first embodiment.

FIG. 2B illustrates an example of the information obtained by the information processing apparatus according to the first embodiment.

FIG. 3 is a flow chart illustrating an example of processing performed by the information processing apparatus according to the first embodiment.

FIG. 4 is a flow chart illustrating an example of the processing performed by the information processing apparatus according to the first embodiment.

FIG. 5 illustrates an example of the information obtained by the information processing apparatus according to the first embodiment.

FIG. 6 illustrates an example of the information obtained by the information processing apparatus according to the first embodiment.

FIG. 7 is a flow chart illustrating an example of the processing performed by the information processing apparatus according to the first embodiment.

FIG. 8 is a flow chart illustrating an example of the processing performed by the information processing apparatus according to a second embodiment.

FIG. 9 illustrates an example of a screen displayed on a display unit by the information processing apparatus according to an embodiment of the present invention.

FIG. 10 illustrates an example of a configuration of a system including the information processing apparatus according to the embodiment of the present invention.

FIG. 11 illustrates an example of a hardware configuration of the information processing apparatus according to the embodiment of the present invention.

### DESCRIPTION OF EMBODIMENTS

Hereinafter, embodiments of the present invention will be described with reference to the drawings.

### First Embodiment

In this specification, an acoustic wave generated by expansion inside a subject when the subject is irradiated with light will be referred to as a photoacoustic wave.

As a method of imaging an internal state of a subject in a low invasive manner, photoacoustic imaging attracts attention. According to the photoacoustic imaging, a living matter is irradiated with pulsed light generated from a light source, and a photoacoustic wave generated from a living tissue that has absorbed energy of the pulsed light propagated and diffused in the living matter is detected. An image obtained by the imaging using the photoacoustic wave will be hereinafter referred to as a photoacoustic image. According to the photoacoustic imaging, an elastic wave (photoacoustic wave) that is generated when a subject site absorbs energy of the irradiated light and momentarily expands by using a difference in an absorption rate of light energy between the subject site such as a tumor and other tissues is received by a transducer. This detected signal will be hereinafter referred to as a photoacoustic signal. A photoacoustic imaging apparatus can obtain an optical characteristic distribution inside the living matter, in particular, a light energy absorption density distribution, by performing analysis processing of the photoacoustic signal. The photoacoustic image includes various images in accordance with an optical characteristic inside the subject. For example, the photoacoustic image includes an absorption coefficient image indicating an absorption density distribution. In addition, an image indicating the presence of biomolecules such as oxygenated hemoglobin, reduced hemoglobin, water, fat, and collagen, a ratio, or the like is generated from the absorption coefficient image. For example, an image related to an oxygen saturation corresponding to an index indicating an oxygen binding state of hemoglobin is obtained on the basis of a ratio between oxygenated hemoglobin and reduced hemoglobin.

In recent years too, a medical image used for a diagnosis and various information related to the diagnosis including the above-described photoacoustic image have been computerized. For example, Digital Imaging and Communications in Medicine (DICOM) standards are used in many cases for information coordination between an imaging apparatus and various apparatuses connected to the imaging apparatus. The DICOM is the standards for defining formats of the medical images and communication protocols between the apparatuses that deal with those images. Data set as a target to be exchanged on the basis of the DICOM is referred to as information object (IOD: Information Object Definitions). Hereinafter, the information object may be referred to as IOD or object in some cases. Examples of the IOD include the medical image, patient information, examination information, structured report, and the like, and various data related to the examination using the medical image and treatment may be set as the target.

The image dealt with on the basis of the DICOM, that is, the image corresponding to the IOD is constituted by meta data and image data. The meta data includes, for example, information related to a patient, an examination, a series, and an image. The meta data is constituted by a set of data elements called DICOM data elements. A tag for identifying the data element is added to each of the DICOM data element. A tag indicating the image data is added to pixel data (image data) corresponding to an example of the DICOM data element. For example, a tag indicating a patient name is added to each of the meta data such as a patient name. In a case where the meta data and the image data are set as a DICOM data set, the IOD may further include DICOM file meta information with respect to the DICOM data set. The DICOM file meta information includes, for example, information of an application that creates the IOD (DICOM file).

To use the photoacoustic image by various apparatuses in a medical facility, the photoacoustic image is also preferably output as the IOD from the photoacoustic imaging apparatus in conformity to the DICOM standards. According to the photoacoustic imaging, as described above, various photoacoustic images can be obtained from the photoacoustic signal related to the single capturing, but when all of the plurality of obtained photoacoustic images are saved or communicated, there is a fear that a capacity related to the saving or the communication is squeezed. According to a first embodiment, it is aimed at outputting the IOD such that the capacity related to the saving or the communication can be reduced by using the meta data related to the photoacoustic image.

Configuration of Information Processing Apparatus

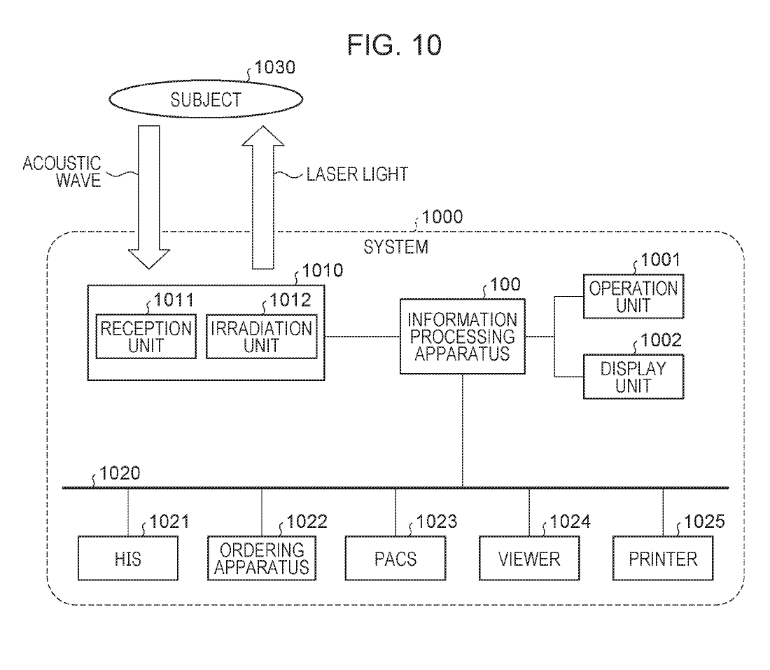

FIG. 10 illustrates an example of a configuration of a system 1000 including an information processing apparatus 100 according to the first embodiment. The system 1000 includes the information processing apparatus 100 and a photoacoustic imaging apparatus 1010. An operation unit 1001 and a display unit 1002 are connected to the information processing apparatus 100. The information processing apparatus 100 is connected to a network 1020. An HIS 1021, an ordering apparatus 1022, a PACS 1023, a Viewer 1024, and a Printer 1025 are connected to the network 1020. The system 1000 is, for example, an information system in a medical facility. The respective components included in the system 1000 do not necessarily need to be installed in the same facility, and it is sufficient when the respective components are connected to one another so as to be communicable.

The information processing apparatus 100 obtains the information related to the examination including the capturing of the photoacoustic image from the ordering apparatus 1022 and controls the photoacoustic imaging apparatus 1010 (hereinafter, referred to as the imaging apparatus 1010) when the above-described examination is performed. The information processing apparatus 100 obtains the photoacoustic signal from the imaging apparatus 1010. The information processing apparatus 100 obtains the photoacoustic image on the basis of the photoacoustic signal. The information processing apparatus 100 performs transmission and reception of information with an external apparatus such as the HIS 1021, the ordering apparatus 1022, or the PACS 1023 in conformity to standards such as Health level 7 (HL7) and Digital Imaging and Communications in Medicine (DICOM).

The operation unit 1001 transmits information related to an operation input by a user to the information processing apparatus 100. The operation unit 1001 is, for example, a key board or a track ball or various buttons for performing operation inputs related to the examination.

The display unit 1002 displays the image obtained from the imaging in the system 1000 and the information related to the examination on the basis of the control from the information processing apparatus 100. The display unit 1002 provides an interface configured to accept user instruction on the basis of the control from the information processing apparatus 100. The display unit 1002 is, for example, a liquid crystal display.

It should be noted that the display unit 1002 and the operation unit 1001 may also be integrated with each other as a touch panel display. In addition, the information processing apparatus 100, the display unit 1002, and the operation unit 1001 do not necessarily need to be separate apparatuses and may be realized as a console in which these configurations are integrated with one another. The information processing apparatus 100 may also include a plurality of probes.

The imaging apparatus 1010 obtains the photoacoustic signal by the technique of the photoacoustic imaging. Regions in the subject set as the targets are, for example, regions such as a cardiovascular region, breasts, neck, abdomen, and extremities including fingers and toes. In particular, a vascular region including plaque of a new blood vessel and a blood vessel wall may also be set as the targets of the imaging of the photoacoustic image in accordance with the characteristic related to the light absorption inside the subject. In the system 1000, for example, imaging to obtain a photoacoustic image of a subject 1030 who has an administration of a dye such as methylene blue or indocyanine green or small gold particles, or a substance obtained by integrating or chemically modifying those as a contrast agent may also be performed.

The imaging apparatus 1010 includes an irradiation unit 1012 that irradiates the subject 1030 with light and a reception unit 1011 that receives the photoacoustic wave from the subject 1030.

The reception unit 1011 includes at least one transducer (not illustrated), a matching layer (not illustrated), a damper (not illustrated), and an acoustic lens (not illustrated). The transducer (not illustrated) is composed of a substance indicating a piezoelectric effect such as lead zirconate titanate (PZT) or polyvinylidene difluoride (PVDF). The transducer (not illustrated) may be an element other than a piezoelectric element and is, for example, a capacitive transducer (CMUT: capacitive micro-machined ultrasonic transducers) or a transducer using a Fabry-Perot interferometer. Typically, the photoacoustic signal is composed of a frequency component at 0.1 to 100 MHz, and the transducer (not illustrated) that can detect these frequencies is used, for example. The signal obtained by the transducer (not illustrated) is a time-resolved signal. An amplitude of the received signal represents a value based on an acoustic pressure received by the transducer at each time. The reception unit 1011 includes a circuit (not illustrated) for an electronic focus or a control unit. An array of the transducers (not illustrated) is, for example, a sector linear array, a convex annular array, or a matrix array. The reception unit 1011 may be provided with an amplifier (not illustrated) configured to amplify a time-series analog signal received by the transducer (not illustrated).

The irradiation unit 1012 includes a light source (not illustrated) arranged to obtain the photoacoustic signal and an optical system (not illustrated) arranged to guide the pulsed light emitted from the light source (not illustrated) to the subject. A pulse width of the light emitted from the light source (not illustrated) is, for example, a pulse width higher than or equal to 1 ns and lower than or equal to 100 ns. In addition, a wavelength of the light emitted from the light source (not illustrated) is, for example, a wavelength higher than or equal to 400 nm and lower than or equal to 1600 nm. In a case where imaging of a blood vessel in the vicinity of a surface of the subject is performed in a high resolution, a wavelength higher than or equal to 400 nm and lower than or equal to 700 nm where the absorption in the blood vessel is large is preferably used. In addition, in a case where imaging of a deep section of the subject is performed, a wavelength higher than or equal to 700 nm and lower than or equal to 1100 nm where the absorption hardly occurs in a tissue such as water or fat is preferably used.

The light source (not illustrated) is laser or a light emitting diode, for example. The irradiation unit 1012 may also use a light source that can convert a wavelength by using light having a plurality of wavelengths to obtain the photoacoustic signal. As an alternative to the above-described configuration, a configuration may be adopted in which the irradiation unit 1012 is provided with a plurality of light sources configured to generate light having mutually different wavelengths and can emit the light having the mutually different wavelengths from the respective light sources. The laser is, for example, solid laser, gas laser, dye laser, or semiconductor laser. Pulsed laser such as Nd:YAG laser or alexandrite laser may be used as the light source (not illustrated). In addition, Ti:sa laser or optical parametric oscillators (OPO) laser in which light of the Nd:YAG laser is set as excitation light may be used as the light source (not illustrated). In addition, a microwave source may be used as the light source (not illustrated).

An optical element such as a lens, a mirror, or an optical fiber is used as the optical system (not illustrated). In a case where the subject is breast, since the irradiation is preferably performed by increasing a beam diameter of the pulsed light, the optical system (not illustrated) may also be provided with a diffused plate that diffuses the emitted light. As an alternative to the above-described configuration, a configuration may be adopted in which the optical system (not illustrated) is provided with a lens or the like and can focus beam to increase the resolution.

The imaging apparatus 1010 converts an analog signal of the photoacoustic wave received by the reception unit 1011 into a photoacoustic signal corresponding to a digital signal to be transmitted to the information processing apparatus 100.

The Hospital Information System (HIS) 1021 is a system for assisting operations in a hospital. The HIS includes an electronic medical record system, an ordering system, and a medical accounting system. When information indicating that the examination is completed is received from the information processing apparatus 100 or the ordering apparatus 1022, the HIS 1021 performs processing for accounting.

The ordering apparatus 1022 is a system where the examination information is managed to manage progresses of the respective examinations of the imaging apparatus. The ordering apparatus 1022 in a radiology department is Radiology Information System (RIS), for example. The examination information includes an examination ID for uniquely identifying the examination and information related to a capturing technique included in the examination. The ordering apparatus 1022 transmits the information of the examination performed by the imaging apparatus 1010 to the information processing apparatus 100 in accordance with a query from the information processing apparatus 100. The ordering apparatus 1022 receives information related to the progress of the examination from the information processing apparatus 100.

A procedure from an examination order issuance to accounting is managed in coordination with one another by the HIS 1021 and the ordering apparatus 1022.

The Picture Archiving and Communication System (PACS) 1023 is a database system where images obtained by various imaging apparatuses inside or outside the facility are held. The PACS 1023 is a PACS server, for example. The PACS 1023 includes a storage unit (not illustrated) configured to store a medical image and accompanied information such as a capturing condition of the medical image, a parameter of image processing including reconstruction, and patient information and a controller (not illustrated) configured to manage the information stored in the storage unit. The PACS 1023 stores an ultrasonic image, a photoacoustic image, or an overlapped image corresponding to an object output from the information processing apparatus 100. A communication between the PACS 1023 and the information processing apparatus 100 and the various images stored in the PACS 1023 are preferably in conformity to the standards such as the HL7 or the DICOM. The various images output from the information processing apparatus 100 are stored while the accompanied information is associated with various tags in conformity to the DICOM standards.

The Viewer 1024 is a terminal for an image diagnosis and reads out the image stored in the PACS 1023 or the like to be displayed for the diagnosis. A doctor displays the image on the Viewer 1024 to observe and records information obtained as a result of the observation as an image diagnosis report. The image diagnosis report created by using the Viewer 1024 may be stored in the Viewer 1024 or output to the PACS 1023 or a report server (not illustrated) to be stored.

The Printer 1025 prints the image stored in the PACS 1023 or the like. The Printer 1025 is a film printer, for example, and prints the image stored in the PACS 1023 or the like on a film to be output.

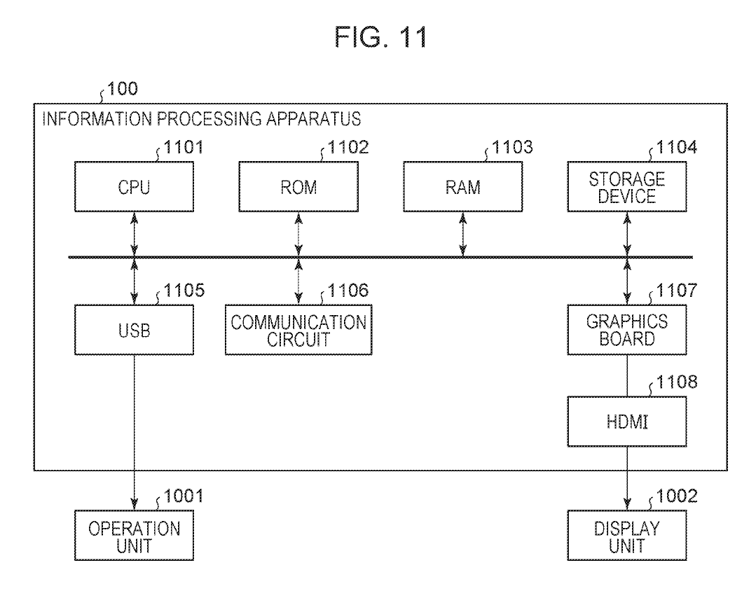

FIG. 11 illustrates an example of a hardware configuration of the information processing apparatus 100. The information processing apparatus 100 is a computer, for example. The information processing apparatus 100 includes a CPU 1101, a ROM 1102, a RAM 1103, a storage device 1104, a USB 1105, a communication circuit 1106, and a graphics board 1107. These are connected to one another via a BUS so as to be communicable. The BUS is used for transmission and reception of data between connected hardware and transmission of a command from the CPU 1101 to the other hardware.

The central processing unit (CPU) 1101 is a control circuit configured to control the information processing apparatus 100 and the respective units connected to the information processing apparatus 100 in an integrated manner. The CPU 1101 implements control by executing a program stored in the ROM 1102. The CPU 1101 also executes a display driver corresponding to software configured to control the display unit 1002 and performs display control with respect to the display unit 1002. Furthermore, the CPU 1101 performs input and output control with respect to the operation unit 1001.

The read only memory (ROM) 1102 stores a program that stores a procedure of the control by the CPU 1101 and data. The ROM 1102 stores a boot program of the information processing apparatus 100 and various pieces of initial data. In addition, the ROM 1102 stores various programs for realizing the processing of the information processing apparatus 100.

The random access memory (RAM) 1103 is configured to provide a working storage area when control based on a command program is performed by the CPU 1101. The RAM 1103 includes a stack and a work area. The RAM 1103 stores programs for executing the processes in the information processing apparatus 100 and the respective units connected to this and various parameters used in the image processing. The RAM 1103 stores a control program to be executed by the CPU 1101 and temporarily stores various pieces of data when the CPU 1101 performs various types of control.

The storage device 1104 is an auxiliary storage device configured to save various pieces of data such as the ultrasonic image and the photoacoustic image. The storage device 1104 is, for example, a hard disk drive (HDD) or a solid state drive (SSD).

The universal serial bus (USB) 1105 is a connection unit to which the operation unit 1001 is connected.

The communication circuit 1106 is a circuit configured to perform a communication with the respective units that constitute the system 1000 and various external apparatuses connected to the network 1020. The communication circuit 1106 stores the information to be output in a transfer packet and performs the output to the external apparatus via the network 1020 by a communication technology such as TCP/IP, for example. The information processing apparatus 100 may also include a plurality of communication circuits in accordance with a desired communication mode.

The graphics board 1107 includes a graphics processing unit (GPU) and a video memory. The GPU performs a calculation related to reconstruction processing for generating the photoacoustic image from the photoacoustic signal, for example.

High-Definition Multimedia Interface (HDMI) (registered trademark) 1108 is a connection unit to which the display unit 1002 is connected.

The CPU 1101 or the GPU is an example of a processor. In addition, the ROM 1102, the RAM 1103, or the storage device 1104 is an example of a memory. The information processing apparatus 100 may include a plurality of processors. According to the first embodiment, when the processor of the information processing apparatus 100 executes a program stored in the memory, functions of the respective units of the information processing apparatus 100 are realized.

In addition, the information processing apparatus 100 may also include a CPU, a GPU, or an application specific integrated circuit (ASIC) that dedicatedly performs particular processing. The information processing apparatus 100 may also include a field-programmable gate array (FPGA) in which particular processing or all processes are programmed.

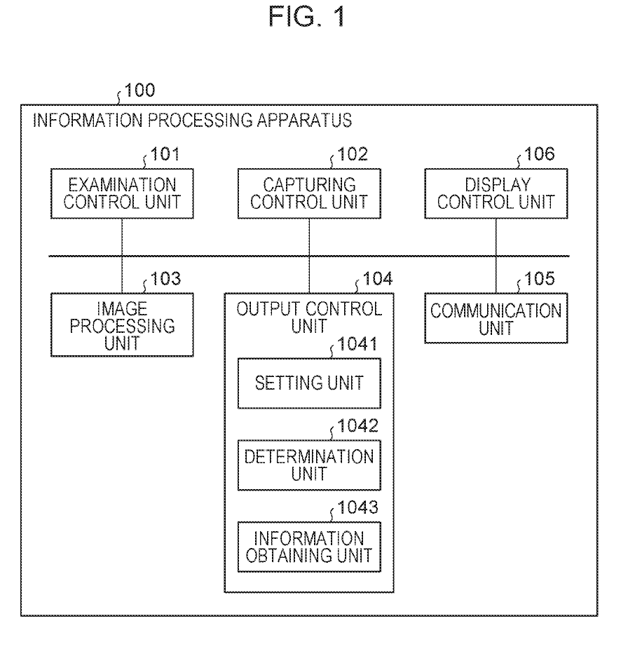

FIG. 1 illustrates an example of a functional configuration of the information processing apparatus 100. The information processing apparatus 100 includes an examination control unit 101, a capturing control unit 102, an image processing unit 103, an output control unit 104, a communication unit 105, and a display control unit 106.

The examination control unit 101 obtains the information of the examination order from the ordering apparatus 1022. The examination order includes the information of the patient subjected to the examination and the information related to the capturing technique. In addition, the examination order may include the information indicating the type of the photoacoustic image which is requested by the user such as the doctor among the photoacoustic images of the plurality of types generated by the photoacoustic signal. The examination control unit 101 transmits the information related to the examination order to the capturing control unit 102. In addition, the examination control unit 101 causes the display unit 1002 to displays the information of the examination to present the information related to the examination to the user via the display control unit 106. The information of the examination displayed on the display unit 1002 includes the information of the patient subjected to the examination, the information of the capturing technique included in the examination, and the already generated image after the completion of the imaging. Furthermore, the examination control unit 101 transmits the information of the progress of the examination to the ordering apparatus 1022 via the communication unit 105.

The capturing control unit 102 controls the imaging apparatus 1010 on the basis of the information of the capturing technique which is received from the examination control unit 101 and obtains the photoacoustic signal from the imaging apparatus 1010. The capturing control unit 102 executes an instruction to the irradiation unit 1012 and an instruction to the reception unit 1011 on the basis of the operation input by the user and the information of the capturing technique. The capturing control unit 102 instructs the irradiation unit 1012 to perform light irradiation. In addition, the capturing control unit 102 instructs the reception unit 1011 to receive the photoacoustic wave. The capturing control unit 102 receives the photoacoustic signal that has been converted into the digital signal in the imaging apparatus 1010. That is, the capturing control unit 102 is an example of a signal obtaining unit configured to obtain the photoacoustic signal.

The image processing unit 103 generates a photoacoustic image. In addition, the image processing unit 103 generates a moving image composed of the photoacoustic image.

Specifically, the image processing unit 103 generates the photoacoustic image on the basis of the photoacoustic signal obtained by the capturing control unit 102. The image processing unit 103 reconstructs a distribution of the acoustic wave at the time of the light irradiation (hereinafter, referred to as an initial acoustic pressure distribution) on the basis of the photoacoustic signal. The image processing unit 103 obtains an intensity distribution of the light with which the subject is irradiated in advance and obtains information related to a light fluence by the following way of thinking, for example.

The image processing unit 103 can calculate a spatial distribution of the light fluence inside the subject by a method of numerically solving a transport equation or a diffusion equation indicating a behavior of the light energy in a medium that absorbs or scatters light. A finite element method, a difference method, a Monte Carlo method, or the like may be adopted as the numerically solving method. For example, the image processing unit 103 may also calculate the spatial distribution of the light fluence (a light fluence distribution, a light amount distribution) inside the subject by solving a related-art light diffusion equation.

The image processing unit 103 obtains an absorption coefficient distribution of light in the subject by dividing the reconstructed initial acoustic pressure distribution by the light fluence distribution (light amount distribution) of the subject with regard to the light with which the subject is irradiated. In addition, the density distribution of the substance in the subject is obtained from an absorption coefficient distribution with respect to a plurality of wavelengths in accordance with the wavelength of the light with which the subject is irradiated by using different light absorption degrees in the subject. For example, the image processing unit 103 obtains the density distribution of the substance in the subject with regard to oxyhemoglobin and deoxyhemoglobin. Furthermore, the image processing unit 103 obtains an oxygen saturation distribution as a ratio of an oxyhemoglobin density to a deoxyhemoglobin density. The photoacoustic image generated by the image processing unit 103 is, for example, an image indicating at least one piece of information of the initial acoustic pressure distribution, the light fluence distribution, the absorption coefficient distribution, the density distribution of the substance, and the oxygen saturation distribution described above. The image related to each of the absorption coefficient distribution, the density distribution of the substance, and the oxygen saturation distribution is generated on the basis of the images related to the initial acoustic pressure distribution and the light fluence distribution as described above. In addition, the image related to each of the density distribution of the substance and the oxygen saturation distribution is generated on the basis of the image related to the absorption coefficient distribution for two wavelengths. That is, the image processing unit 103 obtains the photoacoustic images of the plurality of types. The image processing unit 103 is an example of an image obtaining unit configured to obtain the photoacoustic image.

The output control unit 104 obtains information of the generation of the meta data related to the image and generates an object (hereinafter, referred to as a DICOM object in some cases) to be output to the external apparatus. The output control unit 104 includes a setting unit 1041, a determination unit 1042, and an information obtaining unit 1043.

The setting unit 1041 reads out and obtains the information that should be output as the DICOM object and the setting related to the type of the image data from the memory such as the RAM 1103. As described in the explanation of the image processing unit 103, photoacoustic images of some types among the photoacoustic images of the plurality of types generated on the basis of a certain set of photoacoustic signals are generated on the basis of the other photoacoustic image. For example, the image related to the density distribution of the substance is generated on the basis of the image related to the absorption coefficient distribution for a plurality of wavelengths. The setting unit 1041 stores the information related to the photoacoustic image of the type necessary for generating a photoacoustic image of a certain type. The setting may be input by the user before the examination is performed.

The determination unit 1042 determines whether only the meta data related to the photoacoustic image among the photoacoustic image and the meta data related to the photoacoustic image is output to the external apparatus or the photoacoustic image and the meta data are output to the external apparatus. For example, the determination unit 1042 determines whether or not only the meta data related to the photoacoustic image is output to the external apparatus or the photoacoustic image and the meta data are output to the external apparatus on the basis of the information obtained by at least one of the examination control unit 101 and the capturing control unit 102 and the information stored in the setting unit 1041. The determination unit 1042 performs the determination on the basis of the information related to the photoacoustic image of the type necessary for generating the photoacoustic image which is stored in the setting unit 1041. The determination unit 1042 performs the determination such that the IOD including only the meta data is output with regard to the photoacoustic image of the certain type that can be generated in the external apparatus, and the IOD including the meta data and the photoacoustic image are output with regard to the photoacoustic image of the certain type that is not to be generated in the external apparatus. That is, on the basis of the image related to the type of the photoacoustic image that is to be output to the external apparatus, the determination unit 1042 determines whether or not the photoacoustic image is output to the external apparatus. In a case where it is determined that the photoacoustic image and the meta data are output to the external apparatus, the determination unit 1042 controls the image processing unit 103 to generate the photoacoustic image. The determination unit 1042 is an example of a determination unit.

The information obtaining unit 1043 obtains the DICOM object to be output to the external apparatus. In addition, the information obtaining unit 1043 obtains necessary information for generating the DICOM object. For example, the information obtaining unit 1043 obtains the information related to the obtainment of the photoacoustic image such as the patient information and the examination information from the examination control unit 101, the capturing control unit 102, and the imaging apparatus 1010. The information obtaining unit 1043 obtains the information of the meta data included in the DICOM object. The information obtaining unit 1043 may also obtain the information of the meta data by converting the information that should be written in the meta data into a format in conformity to the DICOM standards. The information obtaining unit 1043 obtains the information of the meta data as a DICOM element. The DICOM element is constituted by an identifier represented in a format of a DICOM tag (gggg, eeee) and information representing contents of data. In addition, the information obtaining unit 1043 obtains the image data of the photoacoustic image generated in the image processing unit 103. The information obtaining unit 1043 is an example of an information obtaining unit.

The communication unit 105 controls the transmission and the reception of the information between the external apparatus such as the HIS 1021, the ordering apparatus 1022, the PACS 1023, or the Viewer 1024 and the information processing apparatus 100 via the network 1020.

The display control unit 106 controls the display unit 1002 to cause the display unit 1002 to display the information. The display control unit 106 causes the display unit 1002 to display the information in accordance with an input from another module or the operation input by the user via the operation unit 1001. The display control unit 106 is an example of a display control unit.

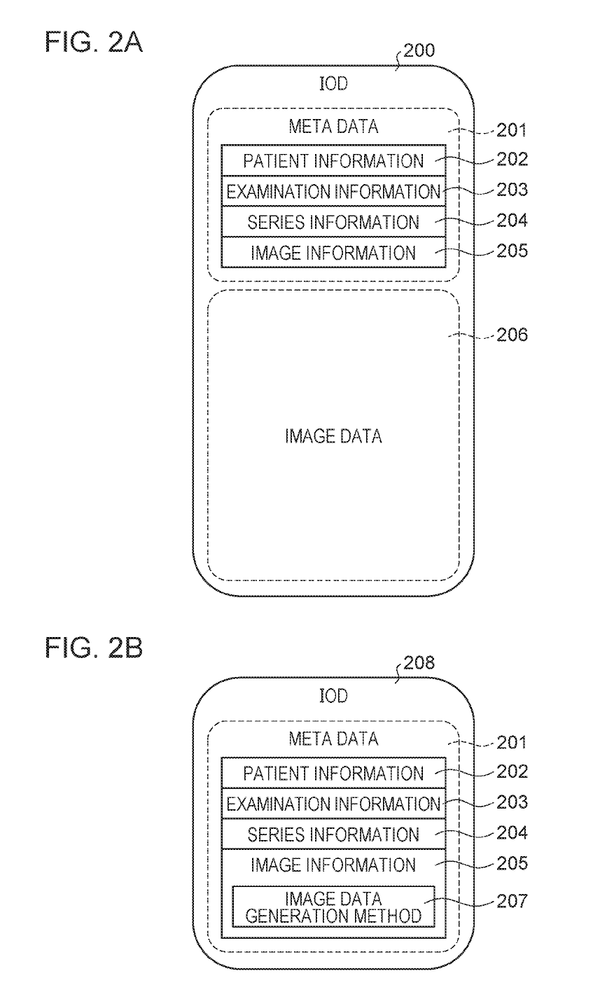

FIGS. 2A and 2B are explanatory diagrams for describing an example of a configuration of the DICOM object obtained by the output control unit 104. FIG. 2A illustrates an example of a configuration of the IOD including the image data and the meta data. An IOD 200 includes meta data 201 and image data 206. The meta data 201 includes patient information 202, examination information 203, series information 204, and image information 205.

The patient information 202 is information of a patient related to the IOD 200 (for example, the subject 1030). The patient information 202 includes information related to a patient ID, a patient name, date of birth, and sex for uniquely identifying the patient, for example.

The examination information 203 is information of an examination related to the IOD 200. The examination information 203 includes, for example, information related to an examination ID (Study Instance UID), an examination date, and an examination time for uniquely identifying the examination.

The series information 204 is information related to the series included in the examination indicated by the examination information 203. The series information 204 includes, for example, a series ID (Series Instance UID) for uniquely identifying the series.

The image information 205 is information related to an image. The image information 205 includes the number of pixels, the number of bits, an orientation of the patient, and an image ID for uniquely identifying the image. The image ID is a service object pair (SOP) Instance UID, for example. In addition, the image information 205 may include the information related to the photoacoustic imaging such as, for example, the presence or absence of the use of the contrast agent.

The image data 206 is pixel data of the image. The image data 206 is generated by the image processing unit 103.

FIG. 2B illustrates the IOD constituted by only the meta data. The IOD 208 does not include the image data 206. The IOD 208 includes an image data generation method 207 corresponding to information related to a method of generating the image data 206. The image data generation method 207 includes the information related to the method for generating the image data related to the IOD 208. A method of generating the image data includes, for example, information for identifying the information necessary for generating the image data and a method for a calculation using the necessary information. In a different perspective, the image data generation method 207 may be regarded as a method with which the image data can be displayed on the Viewer 1024.

Series of Processes Performed by Information Processing Apparatus

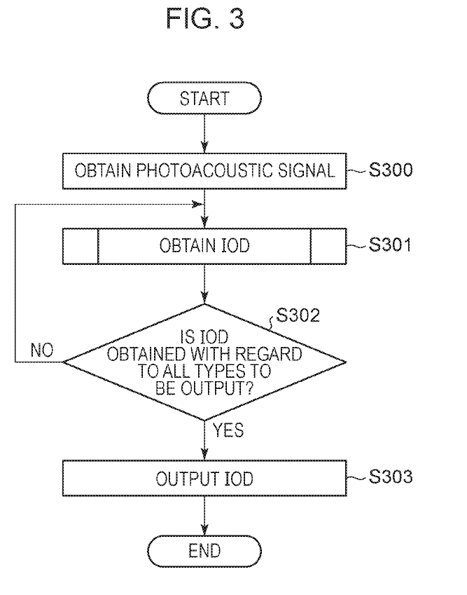

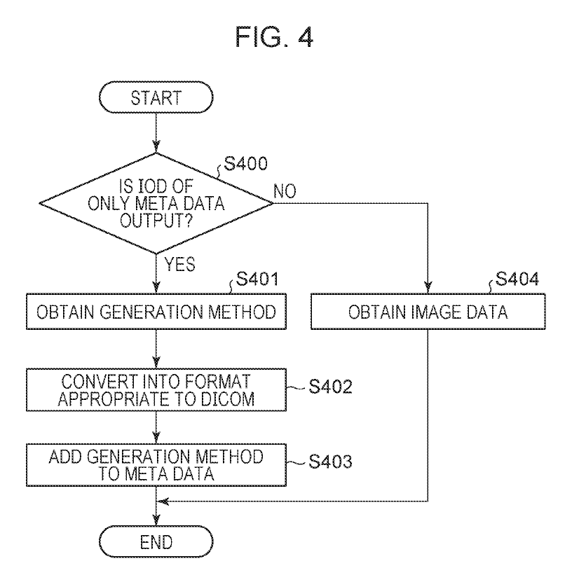

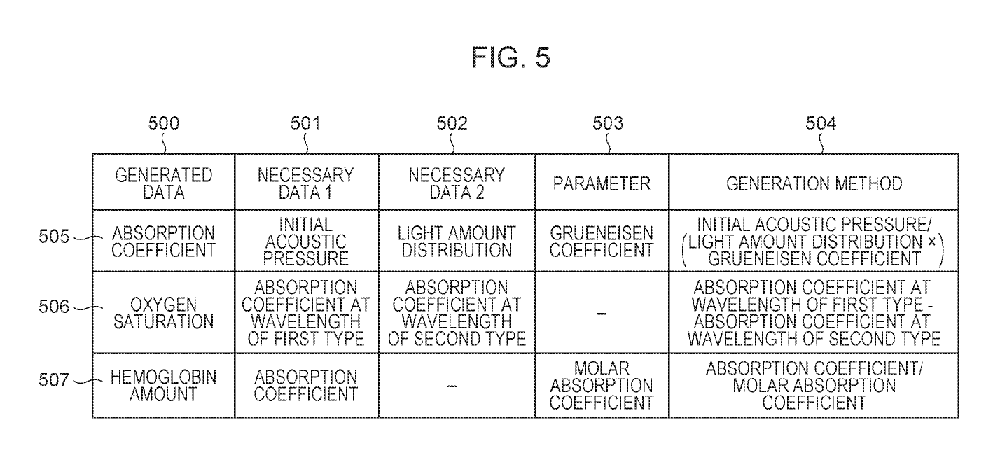

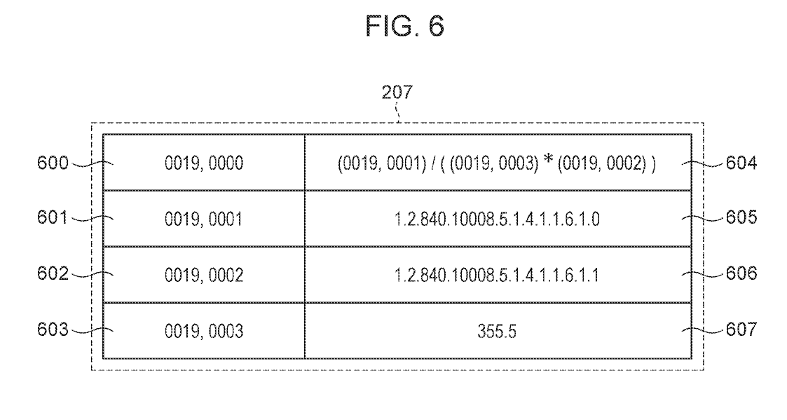

FIG. 3 is a flow chart illustrating an example of a series of processes for the information processing apparatus 100 to obtain the IOD corresponding to an object to be output to the external apparatus. FIG. 4 is a flow chart illustrating an example of a detail of the processing in step S301 illustrated in FIG. 3. In the following processes, unless particularly specified, the subject that realizes the respective processes is the CPU 1101 or the GPU. In addition, FIG. 5 and FIG. 6 respectively illustrate examples of the information obtained by the information processing apparatus 100.

In step S300, the capturing control unit 102 obtains the photoacoustic signal from the imaging apparatus 1010. Hereinafter, a case where the photoacoustic signal measured by using light having two or more different wavelengths is obtained will be described as an example.

In step S301, the output control unit 104 obtains the IOD related to the photoacoustic image generated on the basis of the photoacoustic signal obtained in step S300.

FIG. 4 is a flow chart illustrating an example of detailed processing in step S301.

In step S400, the determination unit 1042 determines a mode in which the DICOM object related to the photoacoustic signal obtained in step S401 is output to the external apparatus on the basis of the information stored in the setting unit 1041. In a case where the determination unit 1042 determines that the IOD including only the meta data (for example, the IOD illustrated in FIG. 2B) is output, the flow proceeds to step S401, and in a case where the determination unit 1042 determines that the IOD including the image data and the meta data (for example, the IOD illustrated in FIG. 2A) is output, the flow proceeds to step S404.

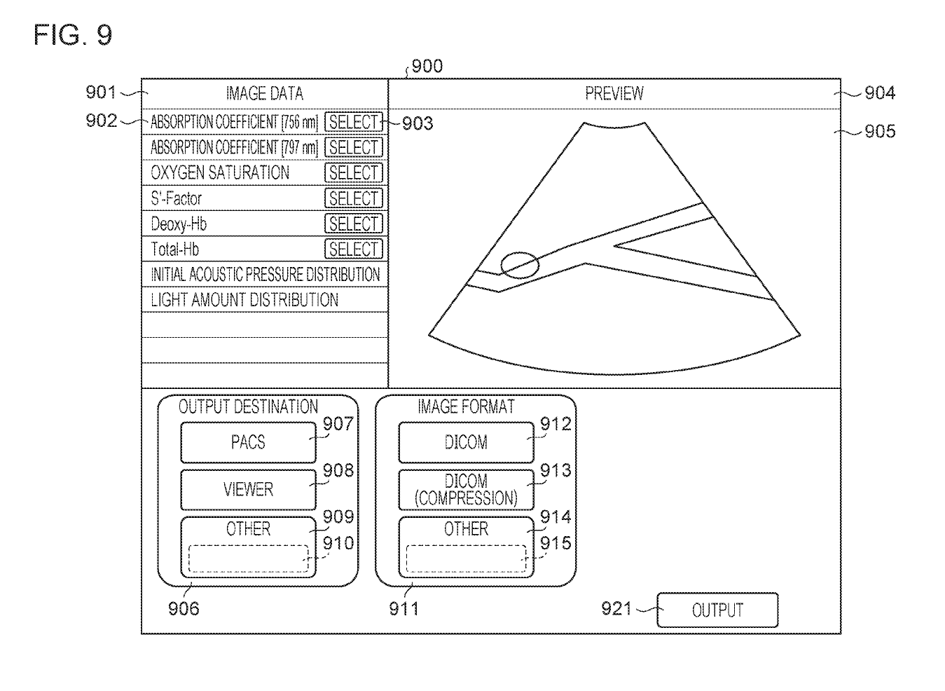

Specifically, the determination unit 1042 obtains the information related to the type of the photoacoustic image obtained in the examination from the examination control unit 101. For example, the determination unit 1042 obtains the information indicating the type of the photoacoustic image that should be obtained included in the order obtained by the examination control unit 101 as the information related to the type of the photoacoustic image obtained in the examination. In addition, the determination unit 1042 may obtain contents of an instruction input by the user via a user interface with regard to the type of the photoacoustic image that should be obtained at the time of the examination. The determination unit 1042 obtains the information related to the type of the photoacoustic image that should be obtained at the time of the examination via the user interface of FIG. 9 which will be described below, for example. It should be noted that, in a case where the type of the photoacoustic image that should be obtained by the order or the input from the user is not particularly specified, the determination unit 1042 may determine that the IODs with regard to all the photoacoustic images that can be obtained are obtained. In addition, the determination unit 1042 may determine that the IOD with regard to only the previously specified type (for example, the image related to the substance inside the subject such as the oxygen saturation) is obtained.

For example, a case will be considered where the order includes information that indicates that the image indicating the initial acoustic pressure distribution, the image indicating the light amount distribution, and the image indicating the absorption coefficient distribution are the photoacoustic images that should be obtained. In this case, the determination unit 1042 determines that the image indicating the absorption coefficient distribution can be generated from the image indicating the initial acoustic pressure distribution and the image indicating the light amount distribution included in the order by referring to a table illustrated in FIG. 5 which will be described below. The determination unit 1042 then determines that, with regard to the image indicating the absorption coefficient distribution, the IOD of only the meta data among the photoacoustic image and the meta data is output to the external apparatus. That is, in a case where a predetermined photoacoustic image included in the photoacoustic images of the plurality of types that should be obtained can be generated by a plurality of other photoacoustic images included in the photoacoustic images of the plurality of types that should be obtained, the determination unit 1042 determines that the IOD of only the meta data among the photoacoustic image and the meta data is output to the external apparatus with regard to the predetermined photoacoustic image. It should be noted that, for example, since the image indicating the initial acoustic pressure distribution and the image indicating the light amount distribution are not described in the table illustrated in FIG. 5, the determination unit 1042 determines that the image indicating the initial acoustic pressure distribution and the image indicating the light amount distribution are not to be generated from the photoacoustic images included in the order. That is, the determination unit 1042 determines that the IOD including the photoacoustic image and the meta data is output to the external apparatus with regard to the image indicating the initial acoustic pressure distribution and the image indicating the light amount distribution.

That is, the determination unit 1042 determines that the image data is not output with regard to a type that can be generated by using the other photoacoustic images to be output among the photoacoustic images of the plurality of types to be output to the external apparatus. When only the image data that is not generated by using the other photoacoustic images is output to the external apparatus, it is possible to reduce the capacity related to the communication and the saving. In a certain perspective, the determination unit 1042 performs the determination with regard to the mode of the IOD such that the number of pieces of image data to be output to the external apparatus is decreased.

In addition, the determination unit 1042 may determine that the IOD including the photoacoustic image and the meta data is output to the external apparatus with regard to the photoacoustic images of the types included in the order. In this case, with regard to the photoacoustic images of the types that are not included in the order, the determination unit 1042 may determine that the IOD including only the meta data among the image data and the meta data is output. According to this, for example, in a case where the user attempts to obtain the IOD of a certain examination by using the Viewer 1024, the IOD including the photoacoustic image of the type requested by the examination order can be obtained and promptly displayed. Furthermore, since the IOD also exists with regard the photoacoustic images of the types that are not requested by the examination order, the Viewer 1024 recognizes that the Viewer 1024 can obtain the photoacoustic images of those types, and the photoacoustic images can be presented to the user as candidates of the image data to be displayed.

Furthermore, similarly in a case where the type of the photoacoustic image that should be obtained is specified via the user interface of FIG. 9 which will be described below, the determination unit 1042 may determine the IOD including only the meta data among the image data and the meta data is output with regard to the photoacoustic image of the type other than the specified type.

The determination unit 1042 obtains the information stored in the setting unit 1041 with regard to each of the types of the photoacoustic images to be output to the external apparatus. The setting unit 1041 stores information related to the photoacoustic image of the type necessary for generating the photoacoustic image of the certain type. In addition, information input via the user interface of FIG. 9 which will be described below is obtained. The determination unit 1042 obtains information indicating whether the output is performed as the IOD including only the meta data to the external apparatus or the output is performed as the IOD including the meta data and the image data to the external apparatus with regard to a certain type of the photoacoustic image. The determination unit 1042 performs the determination on the basis of the information of the setting and determines that the output is performed as the IOD including only the meta data to the external apparatus in a case where the generation can be performed on the basis of the photoacoustic image of the other type. That is, the determination unit 1042 determines whether or not the photoacoustic image of the certain type is output to the external apparatus. In a case where it is determined that the photoacoustic image is output, the control is performed by the following processing such that the IOD including the photoacoustic image and the meta data related to the photoacoustic image is output to the external apparatus. In a case where it is determined that the photoacoustic image is not output, the control is performed by the following processing such that the IOD including only the meta data related to the photoacoustic image is output to the external apparatus.

In step S401, the information obtaining unit 1043 obtains information related to a method of generating the photoacoustic image of the type in which the IOD of only the meta data is output from the setting unit 1041.

FIG. 5 illustrates an example of a method of generating information related to each of the photoacoustic images of the plurality of types (hereinafter, referred to as a generation method). FIG. 5 illustrates an example of the information stored in the setting unit 1041. The generation method is stored in a table format, for example.

A column 500 includes information of “generated data” corresponding to a type of the photoacoustic image set as a target to be generated.

A column 501 and a column 502 include information of “necessary data” corresponding to the type of the photoacoustic image necessary for generating each of the types of the photoacoustic images indicated in the column 500.

A column 503 includes information of parameters necessary for generating each of the types of the photoacoustic images indicated in the column 500.

A column 504 includes information of a “generation method” for generating each of the types of the photoacoustic images indicated in the column 500. The “generation method” indicates a method for generating the photoacoustic image in the column 500 by using the information described in the column 501 to the column 503.

A row 505 indicates, for example, necessary data for generating the image related to the absorption coefficient, a parameter, and a generation method. Specifically, it is indicated that the image related to the initial acoustic pressure and the image related to the light amount distribution (light fluence distribution) are required to generate the image related to the absorption coefficient, and a Grueneisen coefficient is required as the parameter. It is also indicated that the image related to the absorption coefficient is obtained by dividing the data of the initial acoustic pressure by a multiplication result of the data of the light amount distribution and the Grueneisen coefficient.

A row 506 indicates information necessary for generating the image related to the oxygen saturation. A row 507 indicates information necessary for generating the image related to the hemoglobin amount. It should be noted that the mode for storing the generation method is not limited to the example illustrated in FIG. 5.

In step S402, the information obtaining unit 1043 obtains information for describing the information related to the generation method which is obtained step S401 in the meta data of the IOD. Specifically, the information obtaining unit 1043 converts the information related to the generation method into a format in conformity to the DICOM standards.

FIG. 6 illustrates an example of a case where the information related to the generation method is converted into the format in conformity to the DICOM standards. Herein, an example will be described in which the generation method for the image related to the absorption coefficient (the row 505 in FIG. 5) is indicated by a tag and its value.

A tag 600 is a private tag corresponding to the generation method for the image related to the absorption coefficient. A tag 601 and a tag 602 are private tags corresponding to the data necessary for generating the image related to the absorption coefficient. A tag 603 is a private tag corresponding to the parameter for generating the image related to the absorption coefficient.

A value 604 is a value of the tag 600 and indicates a calculation performed by using various data necessary for generating the image related to the absorption coefficient. It is sufficient when the value 604 is the information for the external apparatus to obtain the generation method, and information other than the calculation may also be used. For example, the value 604 may be information for identifying the calculation for generating the image data set as the target, may be information of a path at a saving destination where the information related to the generation method is saved, or may be information at a link destination.

A value 605 and a value 606 are respectively a value of the tag 601 and a value of the tag 602 and indicate information for uniquely identifying the respective pieces of necessary data. The value 605 and the value 606 are, for example, SOP instance UIDs of the necessary data, and the apparatus that has obtained the IOD 208 can obtain the necessary data from the information processing apparatus 100 and the PACS 1023 on the basis of the SOP instance UIDs. It is sufficient when the value 605 and the value 606 are the information for obtaining the necessary data and may be, for example, information of a path at a saving destination in the PACS 1023 or may be information at a link destination.

A value 607 is a value of the tag 603 and indicates a value of the parameter. The mode illustrated in FIG. 6 is merely an example, and of course, the present invention is not limited to this.

In step S403, the information obtaining unit 1043 adds the information related to the generation method which is converted into the format appropriate to the DICOM in step S402 as the image data generation method 207 for the meta data 201.

In step S404, the image processing unit 103 obtains the photoacoustic image on the basis of the photoacoustic signal obtained in step S300.

The IOD related to the photoacoustic image of the certain type is obtained by the above-described processing.

In step S302, the output control unit 104 determines whether or not the IODs related to the photoacoustic images of all the types to be output to the external apparatus are obtained. When the IODs related to the photoacoustic images of all the types are obtained, the flow proceeds to step S303. When the type that has not yet been obtained exists, the flow proceeds to step S302, and the above-described processing is repeated.

In step S303, the communication unit 105 outputs the IOD obtained up to step S302 to the external apparatus such as the PACS 1023 or the Viewer 1024. It should be noted that the order of step S302 and step S303 is not limited to the above-described example, and the IODs that have been obtained by the communication unit 105 may be sequentially output to the external apparatus, for example.

FIG. 9 illustrates an example of a screen 900 displayed by the display control unit 106 on the display unit 1002. The screen 900 is displayed by the display control unit 106 on the display unit 1002 while the processing in FIG. 3 is performed, for example. The screen 900 provides a user interface for the user to select the type of the photoacoustic image to be output to the external apparatus, select an apparatus that outputs the IOD, select a format of the image data, or specify a saving period of the image data.

The type of the photoacoustic image is displayed in a column 901. A button 903 for selecting whether or not the photoacoustic image of the type is output to the external apparatus is displayed in a row of a data type 902. The user can select the type of the photoacoustic image to be output to the external apparatus by operation inputs with respect to the respective buttons. The button 903 is displayed in modes in a manner that a case where the button is selected and a case where the button is not selected can be distinguished from each other. In step S301 illustrated in FIG. 3, the output control unit 104 may also obtain the IOD related to the photoacoustic image of the type selected by the operation input with respect to the screen 900.

In the example illustrated in FIG. 9, a setting in which the image data with regard to the initial acoustic pressure distribution and the light amount distribution are output is a default setting, and in the above-described case, a button for selecting to output those pieces of image data is not displayed. That is, in the example illustrated in FIG. 9, it is possible to select whether or not the image data is output to the external apparatus with respect to the photoacoustic image of the type that can be output by the IOD of only the meta data.

The type selected by the button 903 is displayed in an area 904. Preview display of the photoacoustic image of the type selected by the button 903 is performed in an area 905. After the image data is obtained in step S404 illustrated in FIG. 4, the preview display is performed in the area 905.

An area 906 is an area for selecting a target where the IOD with regard to the photoacoustic image of the type selected by the operation input with respect to the button 903 is output. A button 907 is a button for selecting to perform the output to the PACS 1023. A button 908 is a button for selecting to perform the output to the Viewer 1024. A button 909 is a button for selecting the user to arbitrarily select an output destination, and the output destination can be specified by an operation input with respect to an area 910. In step S302 illustrated in FIG. 3, the communication unit 105 may output the IOD to the external apparatus selected by the operation input with respect to the area 904.

An area 911 is an area for specifying the format of the image data with regard to the photoacoustic image of the type selected by the operation input with respect to the button 903. A button 912 is a button for specifying a format of uncompressed image data in conformity to the DICOM standards. A button 913 is a button for specifying a format of the image data (for example, JPEG2000) compressed in conformity to the DICOM standards. A button 914 is a button for the user to arbitrarily select the format, and the format can be specified by an operation input with respect to an area 915.

A button 921 is a button for instructing the output of the IOD with regard to the photoacoustic image of the type selected by the operation input with respect to the button 903.

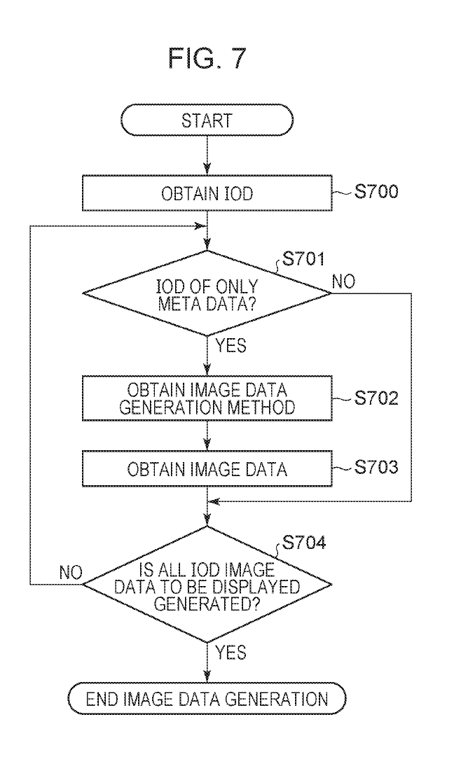

FIG. 7 is a flow chart illustrating an example of processing for displaying the photoacoustic image on the Viewer 1024 on the basis of the IODs obtained in the example illustrated in FIG. 3 to FIG. 6. In the following processes, unless particularly specified, the subject that realizes the respective processes is the CPU 1101 or the GPU. The Viewer 1024 is a computer, for example, and a hardware configuration of the Viewer 1024 is similar to FIG. 11.

In step S700, the Viewer 1024 obtains the IOD from the PACS 1023 or the information processing apparatus 100. That is, the Viewer 1024 receives an object related to the photoacoustic image.

In step S701, the Viewer 1024 determines whether or not the image data is included in the IOD obtained in step S700. In a case where this is the IOD of only the meta data which does not include the image data, the flow proceeds to step S702. In a case where this is the IOD including the image data, the flow proceeds to step S704.

In step S702, the Viewer 1024 obtains information of the image data generation method 207 included in the meta data of the IOD201.

In step S703, the Viewer 1024 obtains the image data indicated by the IOD on the basis of the information of the generation method 207. In a case where the IOD of the necessary data included in the generation method 207 obtained in step S702 is not obtained by the Viewer 1024, the Viewer 1024 obtains the IOD from the PACS 1023 or the information processing apparatus 100. On the other hand, in a case where the obtained IOD is the IOD of only the meta data which does not include the image data, the Viewer 1024 may obtain the information of the generation method 207 similarly as in step S702 described above and obtain the image data on the basis of the generation method. That is, the Viewer 1024 obtains the photoacoustic image on the basis of the meta data of the obtained object.

In step S704, the Viewer 1024 determines whether or not the image data of all of the IODs to be displayed is obtained. In a case where the image data of all of the IODs to be displayed is obtained, the processing illustrated in FIG. 7 is ended. In a case where the IOD where the image data has not yet been obtained among the IODs to be displayed exists, the flow proceeds to step S701, and the above-described processing is repeated.

According to this, the Viewer 1024 can display the image data related to the obtained IOD on a display unit (not illustrated). The Viewer 1024 displays the photoacoustic image on the basis of the meta data of the obtained IOD, in particular, the DICOM tag related to the generation method. The user may install the program of the processing illustrated in FIG. 7 to cause the Viewer 1024 to realize the processing illustrated in FIG. 7.

The processing illustrated in FIG. 7 may be performed at a time point when the IOD is obtained by the Viewer 1024. In addition, the processing may be performed at a time point when the user performs an operation input for viewing image data of a particular IOD via the Viewer 1024. In another example, the user may also set the Viewer 1024 such that the processing is performed at a previously set timing.

According to the configuration of the first embodiment, the information processing apparatus 100 outputs the image data necessary for displaying the image in the external apparatus to the external apparatus and also outputs the meta data that includes the information indicating the generation method for the image data without generating the unnecessary image data. According to this, the capacity of the data output from the information processing apparatus 100 can be reduced, and it is possible to reduce the capacity related to the communication and the capacity related to the saving of the various apparatuses.

### Modified Example of First Embodiment

The setting unit 1041 may further store information of a type desired to be preferentially observed by the user in the external apparatus such as the Viewer 1024. For example, a setting may be performed such that the IOD including the meta data and the photoacoustic image is output to the external apparatus with regard to the photoacoustic image of the type where the user preferentially observes the information related to the density distribution of the substance such as the oxygen saturation. For example, the information processing apparatus 100 outputs the IOD including the image data and the meta data related to the distribution of the substance inside the subject which is used for the diagnosis to the PACS 1023 and outputs the IOD including only the meta data among the image data and the meta data to the PACS 1023 for others. According to this, it is possible to reduce the capacity of the data output from the information processing apparatus 100 to the external apparatus, and also the data used for the diagnosis can be easily displayed in the external apparatus. With regard to the photoacoustic images of types other than the type desired to be preferentially observed, the data with which at least those images can be generated can be generated may be stored in the storage device 1104 of the information processing apparatus 100, and the information related to the generation method or information for the obtainment from the information processing apparatus 100 may be added to the meta data. The information for the obtainment from the information processing apparatus 100 is, for example, information indicating a path of the saving destination of the image data in the information processing apparatus 100. The information obtaining unit 1043 obtains the IOD including the image data and the meta data related to the distribution of the substance inside the subject which is used for the diagnosis among the photoacoustic images of the plurality of different types and obtains the IOD including only the meta data with regard to the photoacoustic image of the other type.

As another example, the determination unit 1042 may also perform the determination in step S400 without using the information of the setting. For example, the determination unit 1042 may perform the determination in step S400 such that the number of pieces of image data to be output to the external apparatus among the types of the photoacoustic images where the output to the external apparatus is requested in the examination order or the like becomes the lowest.

In a case where a combination of the types of the photoacoustic images which is specified by the user is inappropriate as the target to be output to the external apparatus, the output control unit 104 may issue a warning to the user. For example, in a case where the display is not to be performed in the external apparatus unless the image data of all the types is output, that is, in a case where the combination corresponds to a combination where the external apparatus is not to be generated by using the other output image data, the output control unit 104 issues the warning to the user. The output control unit 104 may display a message to the user as the warning on the display unit 1002 via the display control unit 106. The output control unit 104 may also present a combination of types where the image data can be more efficiently output than the combination specified by the user to the user.

In the processing illustrated in FIG. 4, the case has been described as an example where the image processing unit 103 obtains the photoacoustic image in a case where it is determined that not only the meta data among the photoacoustic image and the meta data but also the photoacoustic image are output to the external apparatus. The present invention is not limited to this, and the image processing unit 103 may obtain the photoacoustic image irrespective of the result of the determination in step S400. That is, the processing in step S404 may be performed at any time point before the IOD is output to the external apparatus. In this case, when it is determined in step S400 that only the meta data among the photoacoustic image and the meta data is output to the external apparatus, even if the image processing unit 103 has obtained the photoacoustic image, the output control unit 104 does not output the photoacoustic image to the external apparatus.

The case has been described where the image processing unit 103 previously obtains the intensity distribution of the light with which the subject is irradiated and obtains the light fluence distribution on the basis of the light diffusion equation or the like, but the present invention is not limited to this. For example, the image processing unit 103 may obtain the light fluence distribution while it is assumed that the subject is uniformly irradiated with light. In this case, the image processing unit 103 may also obtain the light fluence distribution by further taking attenuation in a depth direction of the subject into account.

### Second Embodiment

According to the first embodiment, the example has been described in which, with regard to the photoacoustic image of the type which can be generated by the external apparatus, the IOD of only the meta data including the information related to the generation method is obtained to be output to the external apparatus as the processing for efficiently outputting the image data from the information processing apparatus 100 to the external apparatus.

According to a second embodiment, a case will be described as an example where the IOD of only the meta data or the IOD including the meta data and the image data is obtained from the information processing apparatus 100, and the image data desired by the user to be displayed is obtained in the storing PACS 1023 to be transmitted.

The PACS 1023 according to the second embodiment includes a functional configuration similar to the image processing unit 103 of the information processing apparatus 100 illustrated in FIG. 1.

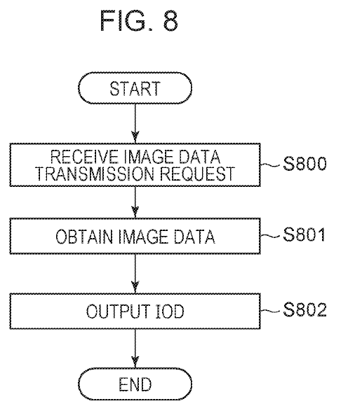

FIG. 8 is a flow chart illustrating an example of the processing performed by the PACS 1023. In the following processes, unless particularly specified, the subject that realizes the respective processes is a processor (not illustrated) of the PACS 1023.

In step S800, the PACS 1023 accepts a transmission request of the image data from the external apparatus such as the Viewer 1024.

In step S801, the PACS 1023 obtains the image data on the basis of the transmission request obtained in step S801. The PACS 1023 searches for the IOD related to the photoacoustic image of the type where the transmission request has been made. In a case where the IOD does not exist in the PACS 1023, the information processing apparatus 100 may be requested to perform the output. In a case where the image data is not included in the searched IOD, the PACS 1023 obtains the image data on the basis of the image data generation method 207 included in the meta data 201. The processing for obtaining the image data on the basis of the generation method 207 is similar to the processing described according to the first embodiment. The detailed descriptions will be omitted here by supporting the above-described explanation.

In step S802, the PACS 1023 outputs the IOD including the image data corresponding to the transmission request received in step S801 to the Viewer 1024.

With the configuration according to the second embodiment, the PACS 1023 can suppress the capacity related to the saving of the data received from the information processing apparatus 100. Then, the image data is obtained to be output to the Viewer 1024 with respect to the transmission request for the display on the Viewer 1024, so that the Viewer 1024 can easily display the image data.

### Modified Example of Second Embodiment

The PACS 1023 may also determine whether the IOD of only the meta data is output or the IOD including the image data and the meta data is output in accordance with the function of the Viewer 1024. For example, in a case where the Viewer 1024 includes the function of the image processing, the IOD of only the meta data may be output to the Viewer 1024 in step S902 without obtaining the image data in step S901.

As another example of the second embodiment, the information processing apparatus 100 may output the IOD related to the photoacoustic image in the following steps, and the PACS 1023 may obtain the IOD.

(A) A step of obtaining first photoacoustic image data (for example, 3D volume data) for the information processing apparatus 100 to form a first photoacoustic image (for example, the initial acoustic pressure distribution image).
(B) A step of obtaining second photoacoustic image data (for example, volume data) for the information processing apparatus 100 to form a second photoacoustic image (for example, the absorption coefficient distribution) by using the first photoacoustic image data and other information (for example, information related to the light fluence distribution).
(C) A step for the information processing apparatus 100 to transmit a first IOD including the first photoacoustic image data and first meta data related to the first photoacoustic image to the PACS 1023. That is, this step is a step for the PACS 1023 to obtain the first IOD.
(D) A step for the information processing apparatus 100 to transmit second meta data including information with which the PACS 1023 can generate second photoacoustic image data on the basis of the first photoacoustic image data to the PACS 1023 even in a case where the PACS 1023 does not hold the second photoacoustic image data. The information mentioned herein is, for example, information related to the light fluence distribution or information related to a calculation expression for estimating a light intensity inside the subject from an intensity of the light from the light source with which the subject is irradiated and a light intensity distribution.

### Modified Examples

According to the above-described embodiments, the processing performed when the photoacoustic image obtained in the photoacoustic imaging apparatus is output as the DICOM object has been described, but the present invention is not limited to this. For example, an ultrasonic image and a photoacoustic image obtained by an imaging apparatus that can obtain a photoacoustic signal and an ultrasonic signal may be set as the targets of the above-described processing. In addition, a computed tomography (CT) image obtained in a CT apparatus or a magnetic resonance imaging (MRI) image obtained in an MRI apparatus may be set as the target of the above-described processing. In the case of the medical image obtained by any imaging apparatus, when the information indicating the generation method is described in the meta data while the image data that can be generated on the basis of certain image data corresponding to the image data of a plurality of types generated on the basis of a certain group of signals is not included in the IOD, the load of the communication for the above-described image data can be reduced. The meta data of the IOD which does not include the image data may include all information except for the image data or may also be constituted by only information to which a required tag is added. It is sufficient when the meta data of the IOD which does not include the image data includes the information indicating the generation method for the image data.

According to the above-described embodiments, the example has been described in which the IOD which includes the image data of the photoacoustic image and the IOD which does not include the image data of the photoacoustic image are output to the external apparatus in accordance with the type of the photoacoustic image, but the present invention is not limited to this. For example, the output control unit 104 may also output the IOD including high-resolution image data of the photoacoustic image and the IOD including simple image data of the photoacoustic image and information related to a method of obtaining the high-resolution image data from the simple image data to the external apparatus in accordance with the type of the photoacoustic image. The external apparatus that has obtained the IOD can read out the meta data and obtain the high-resolution image data from the simple image data on the basis of the other image data and information of numeric values. When the control is performed such that the high-resolution image data is not output with regard to the photoacoustic images of at least part of the types, it is possible to reduce the capacity related to the communication and the saving. In another example, the output control unit 104 may output the IOD including the image data and the meta data of a region of interest (ROI) in the photoacoustic image to the external apparatus, for example. In this case, the output control unit 104 describes information for generating a region other than the ROI in the photoacoustic image in the meta data. The Viewer 1024 can obtain the IOD including the image data in the ROI and easily display the ROI on the display unit (not illustrated) of the Viewer 1024. In addition, when the Viewer 1024 reads out the meta data, it is possible to obtain an image in the region other than the ROI.

The present invention can also be realized by the processing in which a program that realizes one or more functions of the above-described embodiments is supplied to a system or an apparatus via a network or a storage medium, and one or more processors in a computer of the system or the apparatus read out and execute the program. In addition, the present invention can be realized by a circuit that realizes one or more functions (for example, an ASIC).

The information processing apparatus according to the above-described respective embodiments may be realized as a stand-alone apparatus and may also adopt a mode in which a plurality of apparatuses are combined so as to be mutually communicable to execute the above-described processing, both of which are included in the embodiments of the present invention. A common server apparatus or a server group may also execute the above-described processing. It is sufficient when a plurality of apparatuses constituting the information processing apparatus and the information processing system are communicable at a predetermined communication rate and are not required to exist in the same facility or in the same country.

For example, part of the functional configurations illustrated in FIG. 1 may be included in the PACS 1023 instead of the information processing apparatus 100 or may also be included in the Viewer 1024, and the above-described processing may be realized as a system constituted by these apparatuses. For example, the image processing unit 103 and the output control unit 104 may be included in the PACS 1023, and the PACS 1023 may control the mode of the IOD to be output to the Viewer 1024.

The embodiments of the present invention include a mode in which a software program for realizing the functions of the above-described embodiments is supplied to a system or an apparatus, and a computer of the system or the apparatus reads out and executes a code of the supplied the program.

Therefore, in order that the computer realizes the processing according to the embodiment, a program code itself to be installed in the computer is also one of the embodiments of the present invention. In addition, an operating system (OS) or the like running on the computer performs part or all of the actual processes on the basis of an instruction included in the program read out by the computer, and the functions of the above-described embodiments may also be realized by the processing.

A mode obtained by appropriately combining the above-described embodiments is also included in the embodiments of the present invention.

The present invention is not limited to the above-described embodiments, and various modifications and alterations can be made without departing from the spirit and the scope of the present invention. Thus, the following claims are accompanied to make the scope of the present invention public.

According to the present invention, the output of the photoacoustic image to the external apparatus can be omitted in some cases by using the meta data in accordance with the type of the photoacoustic image. In the above-described case, the load in the saving and the communication can be reduced.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information processing apparatus comprising:
a determination unit configured to determine, on a basis of information related to a type of a photoacoustic image generated on a basis of a photoacoustic signal obtained by irradiating a subject with light, whether or not the photoacoustic image is output to an external apparatus; and
an output unit configured to output the photoacoustic image and meta data related to the photoacoustic image to the external apparatus in a case where the determination unit determines that the photoacoustic image is output to the external apparatus, and
selectively output the meta data among the photoacoustic image and the meta data to the external apparatus in a case where the determination unit determines that the photoacoustic image is not output to the external apparatus.

2. The information processing apparatus according to claim 1, wherein the determination unit determines that a photoacoustic image of a type which can be generated on a basis of the photoacoustic image to be output among photoacoustic images of a plurality of types is not output to the external apparatus.

3. The information processing apparatus according to claim 1, wherein the determination unit determines whether or not the photoacoustic image is output to the external apparatus on a basis of methods for respectively generating the photoacoustic images of the plurality of types.

4. The information processing apparatus according to claim 1, wherein the determination unit determines that a photoacoustic image of a type which is used for a diagnosis by a user among photoacoustic images of a plurality of types is output to the external apparatus.

5. The information processing apparatus according to any one of claim 1, wherein the output unit outputs the meta data including information related to a method with which the photoacoustic image can be displayed by the external apparatus to the external apparatus in a case where the determination unit determines that the photoacoustic image is not output to the external apparatus.

6. The information processing apparatus according to claim 5, wherein the method with which the photoacoustic image can be displayed is the method for generating the photoacoustic image.

7. The information processing apparatus according to claim 6, wherein the method with which the photoacoustic image can be displayed is a method for obtaining the photoacoustic image.

8. The information processing apparatus according to claim 1, wherein the output unit outputs information object including the meta data as a DICOM element to the external apparatus.

9. The information processing apparatus according to claim 1, wherein the photoacoustic image is an image representing at least one of an absorption coefficient, an oxygen saturation, and a distribution of a substance in a living matter.

10. The information processing apparatus according to claim 1, further comprising an image obtaining unit configured to obtain a photoacoustic image of at least one type among the photoacoustic images of the plurality of types on a basis of the photoacoustic signal.

11. The information processing apparatus according to claim 1, further comprising a setting unit configured to perform a setting related to a type of a photoacoustic image to be output to the external apparatus among the photoacoustic images of the plurality of types,
wherein the determination unit perform a determination on a basis of the setting.

12. An information processing apparatus comprising:
an image obtaining unit configured to obtain photoacoustic images of a plurality types which are generated on a basis of a photoacoustic signal obtained by irradiating a subject with light;
an information obtaining unit configured to obtain meta data related to the photoacoustic image; and
an output unit configured to selectively output first meta data related to a photoacoustic image of a first type among the plurality of types to an external apparatus and output second meta data related to a photoacoustic image of a second type among the plurality of types and a photoacoustic image of the second type to the external apparatus.

13. The information processing apparatus according to claim 12, wherein the photoacoustic image of the first type is a photoacoustic image related to a distribution of a substance inside the subject.

14. The information processing apparatus according to claim 12, wherein the photoacoustic image of the second type is a photoacoustic image related to a distribution of a substance inside the subject.

15. An information processing method comprising:
determining, on a basis of information related to a type of a photoacoustic image generated on a basis of a photoacoustic signal obtained by irradiating a subject with light, whether or not the photoacoustic image is output to an external apparatus; and
outputting the photoacoustic image and meta data related to the photoacoustic image to the external apparatus in a case where it is determined that the photoacoustic image is output to the external apparatus, and
selectively outputting the meta data among the photoacoustic image and the meta data to the external apparatus in a case where it is determined that the photoacoustic image is not output to the external apparatus.

16. A non-transitory computer-readable medium that stores a program for causing a computer to execute the information processing method according to claim 15.

17. An information processing method comprising:
obtaining meta data related to photoacoustic images of a plurality types which are generated on a basis of a photoacoustic signal obtained by irradiating a subject with light; and
selectively outputting first meta data related to a photoacoustic image of a first type among the plurality of types to an external apparatus and outputting second meta data related to a photoacoustic image of a second type among the plurality of types and a photoacoustic image of the second type to the external apparatus.

18. A non-transitory computer-readable medium that stores a program for causing a computer to execute the information processing method according to claim 17.
